(19) United States
(12) Patent Application Publication
(10) Pub. No.: US 2004/0029270 A1
(43) Pub. Date: Feb. 12, 2004

(54) Vitro human angiogenesis model

(76) Inventors: Lucie Germain, St-Augustin (CA); Francois A Auger, Sillery (CA); Rochon Marie-Helene, Verdun (CA); Annie F Black, Marseille (CA)

Correspondence Address: OGILVY RENAULT, 1981 MCGILL COLLEGE AVENUE, SUITE 1600, MONTREAL, QC, H3A2Y3, CA

(21) Appl. No.: 10/333,919

(22) PCT Filed: Sep. 5, 2001
(86) PCT No.: PCT/CA01/01276

(51) Int. Cl. C12N 5/08

(52) U.S. Cl. 435/371

(57) ABSTRACT

The present invention relates to a tridimensional living tissue construct free of exogenous extracellular matrix material, and in which angiogenesis occurs. Network of capillaries formed into the living tissue construct allows to provide blood vessels for replacement or repair in human and animals, and is also useful in screening compounds or agents potentially having capability of modulating the angiogenesis in vitro or in vivo.

### BACKGROUND OF THE INVENTION

(a) Field of the Invention

The invention relates to tissue engineering and production of tissue constructs. The tissues formed may be used for tissue replacement, repair or for screening and testing of agents potentially activators or inhibitors of angiogenesis.

(b) Description of Prior Art

Angiogenesis, or the development of new blood vessels, is an essential feature of tissue development and wound healing. Without the appropriate development of a blood supply, tissues cannot survive, the circulatory system being essential for the supply of oxygen and nutrients to tissues and for removal of by products of metabolism. Once the vascular system is fully developed, endothelial cells of blood vessels normally remain quiescent with almost no new vessel formation. If disease or injury occurs, the formation of new blood vessels can proceed normally, as in natural wound healing, or be insufficient, as in chronic dermal ulcers, or abnormally upregulated as seen in tumorogenesis, diabetic retinopathy, psoriasis and inflammation. Inhibition of inappropriate angiogenesis or enhancement of angiogenesis in non-healing wounds is therefore an extremely important target for drug discovery programs. However, research in this area leading to new drug development has been hindered by the lack of representative in vitro models of angiogenesis.

In mature tissues, angiogenesis has a relatively low occurrence except during wound healing and the female menstrual cycle. However, there are a number of “angiogenesis-dependent diseases” in adults where angiogenesis is of critical importance. The growth of solid tumors, and the onset of diabetic retinopathies or rheumatoid arthritis rely on the upregulation of angiogenesis.

At early stages of tumour growth, cancer cells are not able to recruit surrounding capillaries and to augment the blood flow to sustain their growth. These cells must thus acquire the ability to elicit and sustain a capillary network that surrounds and nourishes the tumour. This process is known as the angiogenic switch. Once cancer cells are able to sustain a robust angiogenic response around the tumour, their proliferation is accelerated, and the invasion of other tissues may then begin by metastasis.

In order to activate quiescent endothelial cells, it has been observed that cancer cells must be able to efficiently modulate different elements of the angiogenic balance, thus leading to capillary sprouting. Growth factors release from the tumour, modulation or cleavage of proteins from the extracellular matrix, upregulation of proteinase expression are thought to play an important role in the development of a capillary network necessary for the sustained proliferation of cancer cells.

To alter the progression of various solid tumours, many treatments are currently being designed to halt tumour angiogenesis, thereby limiting the growth of the cancer cells. These treatments can intervene on different elements implicated in the angiogenic balance to limit endothelial cell proliferation. This avenue should be beneficial in the treatment of cancers, and some treatments are already in clinical trials (Hagedorn and Bikfalvi, 2000, Crit Rev Oncol Hematol 34:89-110).

The development of angiogenesis inhibitors may provide a mean for controlling these diseases but current assays for angiogenesis are cumbersome, time consuming and usually based on in vivo systems. The three most frequently used models are the rabbit corneal pocket, the hamster cheek pouch and the chicken chorioallantoic membrane (CAM) assays. In each system an angiogenic substance must be implanted in the cornea, cheek pouch or the CAM in order to induce angiogenesis. All three assays have hampered in their clinical relevance by need to artificially induce angiogenesis, This being the requirement for a sustained-release carrier substance vehicle for the angiogenic substance and inhibitor, and the technical complexities associated with setting up the assay, including using live animals, and measuring the outcome. Moreover these assays have the limits of animal model that may behave differently from human cells. The rabbit corneal assay has the additional disadvantage of being ethically now debatable in many research institutions.

Different in vitro models of angiogenesis have been devised and utilized in order to help analyse the different steps implicated in capillary sprouting.

These models are composed of cells, most of the time comprising only one cell type obtained from animals (Nehls et al., 1994, Microvasc Res 48:349-363), contain extracellular matrix components of non-human origin or only one element of the extracellular matrix. Although these models help to understand the cellular and molecular basis of angiogenesis, they are too simplified to fully mimic the in vivo angiogenesis phenomenon. None of them present adequate ultrastructural features of capillary network harbouring a tridimensional organization.

Because of these disadvantages there is a great need for physiologically relevant cell system capable of being equivalent or highly representative of blood vessels and capillaries in vivo, and for in vitro assays for measuring the potential inhibitory or stimulatory activity of some agents on the angiogenesis, particularly human angiogenesis. Previous in vitro assays have usually entailed establishing long-term cultures of endothelial cells and inducing the formation of microvessels by placing the cells on extracellular matrices or exposing the cells to various angiogenic stimuli. Such assays are highly simplified and may not represent a normal physiological response.

Considering the above state of the art, there are still important needs in providing tridimensional cell culture comprising more than one cell type that can be used for production of reconstructed tissues, which can be used also as tools for evaluating the potential of different substances, cells or agents to act as angiogenic modulators, activators or inhibitors.

### SUMMARY OF THE INVENTION

One object of the present invention is to provide a tridimensional living tissue construct (3DLTC) comprising fibroblasts and endothelial cells cultured under conditions allowing synthesis and self-assembling of an extracellular matrix, the cultured fibroblasts and endothelial cells being embedded into or associated with the extracellular matrix for forming a tridimensional capillary network within the 3DLTC.

The extracellular matrix may be capable of supporting the growth of epithelial cells typs.

The epithelial cells, fibroblasts and endothelial cells can be of human origin, from various blood vessels or precursor sources and the epithelial cells' may be derived from a normal, a pre-malignant or a malignant epithelium.

The fibroblasts and endothelial cells of the tridimensional living tissue construct of the present invention may be further self-assembled into a stroma, and can further comprise epithelial cells in order to form an epithelium.

Another object of the present invention is to provide a method for determining potential angiogenic modulating properties of at least one agent based on measurement of vessels in a capillary network present in a tridimensional living tissue construct (3DLTC) of the present invention, comprising the steps of:

a) contacting at least one agent with a 3DLTC in culture, the agent being dissolved in a cell culture medium or applied onto the 3DLTC for a time sufficient to allow the agent to inhibit or stimulate growth of vessels in the 3DLTC; and

b) measuring growth of vessels of the capillary network in contact with at least one agent,

wherein the measurement of growth comprises determining a growth inhibition or a growth stimulation of said vessels compared to the growth of vessels of a 3DLTC non-treated with the at least one agent.

The agent of the present method may be selected from the group consisting of a peptide, a polypeptide, a protein, a fatty acid, a lipid, sugar, a carbohydrate or a combination thereof. In the method of the invention, the tridimensional living tissue construct may contain epithelial cells derived from a normal, a pre-malignant or a malignant epithelium.

Another object of the present invention is to provide a method for in vitro determination of the potential angiogenic modulating properties of exogenous cells based on growth of vessels in a capillary network present in a 3DLTC of the present invention, the method comprising the steps of:

c) contacting an exogenous cell with the 3DLTC, either applied directly onto the 3DLTC or placed in a same culture medium for a time sufficient to allow the exogenous cell to inhibit or stimulate growth of vessels in the 3DLTC; and

d) measuring growth of vessels of the capillary network of the 3DLTC in contact with the exogenous cell;

wherein the exogenous cells are exogenous with respect to cells contained in the 3DLTC, and an intrinsic ability of the exogenous cell to affect angiogenesis in vivo is reflected in the 3DLTC by stimulating or inhibiting the angiogenesis.

In accordance with the present invention there is also provided a method wherein the tridimensional living tissue construct may contain epithelial cells derived from a normal, a pre-malignant or a malignant epithelium.

For the purpose of the present invention the following terms are defined below.

The terms “neovascularization, vasculogenesis and angiogenesis” are intended to mean the process whereby new blood vessels are formed.

The term “angiogenesis modulation” as used herein means the ability of a substance to modulate or change normal angiogenic activity of the tridimensional living tissue construct (3DLTC) and includes inhibition and enhancement of angiogenic activity. The method may be used to test compounds or substances which are possible angiogenesis inhibitors or possible angiogenesis enhancers.

### BRIEF DESCRIPTION OF THE DRAWINGS

FIGS. 1A and 1B illustrate the histological cross sections of an in vitro angiogenesis tridimentional tissue construct (3DLTC) containing normal (non-malignant) epithelial cells (keratinocytes), fibroblasts and endothelial cells after 21 days of maturation in culture at an air-liquid interface;

FIGS. 2A to 2Q illustrate the immunofluorescence staining of different antigens present in an in vitro angiogenesis 3DLTC containing normal (non-malignant) epithelial cells (keratinocytes), fibroblasts and endothelial cells;

FIGS. 3A to 3C illustrate the ultrastructure of endothelialized tubes found in an in vitro model of angiogenesis, the 3DLTC;

FIGS. 4A to 4F illustrate a confocal microscopy analysis of an in vitro angiogenesis 3DLTC; and

FIGS. 5A to 5E illustrate the histological properties of an in vitro model of angiogenesis, the 3DLTC containing fibroblasts, endothelial cells and different types of epithelial cells.

### DETAILED DESCRIPTION OF THE INVENTION

In accordance with the present invention, there is provided a tridimensional living tissue construct (3DLTC) that can be used both as replacement tissues or as tools for evaluating the potential of different substances and agents as angiogenic modulators, activators or inhibitors.

According to another embodiment of the present invention, there is provided a 3DLTC that is composed by a co-culture of at least one layer formed with fibroblasts and endothelial cells mixed together. The cells may be of different origins. Epithelial cells may be seeded onto this first layer to form another layer. The cells may be normal epithelial cells or cancer cells. The presence of cancer cells as epithelial cells in the 3DLTC provides a system that has the advantage of representing natural conditions in which a cancer cell interacts with surrounding cells and extracellular matrix in an organism.

Another embodiment of the present invention is that the method of producing reconstructed vessels allows for the production of capillaries that have a network organization, where the lumen of vessels communicate together as in a naturally occurring vessel network. In this construct, the cells are involved in the formation of a tridimentional network, for example through gap or tight junction or various other ways; this favors communications as it happens in vivo.

Also, the present invention makes use of cells organized in several layers of at least two cell types instead of only one monolayer as found in the prior art. This organization provides vascular tissues that can be directly introduced in an animal while the graft vessel and endogenous vessels can than connect together. Because of its elaborated cellular composition, network organization and structural compatibility, the connection of the vessels a 3DLTC of the present invention with endogenous vessels that have to be repaired may help its proper occurrence in vivo.

Preferably, the 3DLTC or reconstructed blood vessels provided with the present invention are made of cells derived from various tissues, for example, human tissue, which is readily available such as biopsies and solid tumors. It will be appreciated that the 3DLTC or reconstructed blood vessels of the invention can also be used to assay angiogenesis in cell systems or reconstructed blood vessels as defined in the present invention. It will also be understood from those skilled in the art that the 3DLTC of the present invention can be made of cells originating from other animals or mammals, like pig, cow or primate for example. Alternatively, endothelial cells could be derived from stem cells or precursor cells. Fibroblasts could be isolated from other tissues such as blood vessels or ligament.

One particular embodiment of the present invention provides a 3DLTC that may be entirely composed of human components (cells and extracellular matrix elements), and to which is not added synthetic or exogenous matrix material. This invention therefore offers a microenvironment similar to an in vivo stroma, and it also allows normal, premalignant, and malignant epithelial cells to grow in and/or on a complex reconstructed tissue.

Fibroblasts and keratinocytes may be isolated, but not exclusively, from the skin of human volunteers, endothelial cells from the umbilical vein of a healthy donor, the skin's capillaries of a human donor, or the stromal vessels of any kind of biopsy. Fibroblasts and endothelial cells are co-cultured and grown under optimal conditions in order to recreate for example, but not limited to, a stromal moiety. Fibroblasts and endothelial cells are embedded in a complex tridimensional matrix that they have secreted and assembled. In this reconstructed stroma, the endothelial cells reorganized into a tridimensional network of vascular tubes having a lumen, within the extracellular matrix. Basement membrane proteins may line these hollow tubes. Angiogenic sprouts may also be observed within such a network.

This reconstructed stroma can also support the growth of normal, premalignant and malignant epithelial cells. These epithelial cells may adopt different phenotypes in and/or on the stroma, which may be similar to the observed in vivo carcinogenesis phenomenon. These various 3DLTC allow quantifying the angiogenic potential of the different epithelial cells and also the testing of angiogenic-modulating agents.

In still another embodiment of the present invention, there is provided a method for screening substances or cells having a potential angiogenesis modulation activity.

In one embodiment of the present invention, the method comprises culturing a 3DLTC with at least one substance or an exogenous cell suspected of having angiogenesis modulation activity for a time period and under conditions that are sufficient to stimulate or inhibit the growth of vascular tissues contained into a 3DLTC; to examine and analyze the 3DLTC for new vascular tissue growth or inhibition of growth. The results of growth can be compared to that of a non-treated 3DLTC control. The growth may be considered as the quantity of capillaries formed in the culture conditions for a determined period of time according to various analysis parameters.

In the present method, the 3DLTC is cultured under appropriate conditions to allow the growth of new vascular tissue. Those skilled in the art will be familiar with the various appropriate conditions that may be used to culture the 3DLTC.

Examination of the 3DLTC may be carried out by any convenient means. Preferably examination of the 3DLTC may be carried out by bright field or phase contrast light microscopy. This may be also done also using an inverted microscope or confocal microscope.

The responses of the 3DLTC (angiogenic or otherwise) to different agents can be quantified manually or by computer based image analysis of photographs, video images or digital images of the cultures. Such quantification provides rapid and accurate assessment of the responses. In addition, such an assay system is particularly well suited for screening inhibitors or enhancers of angiogenesis. Selected agents or cells according to the results of the present method can be used for treatments of animal or human cancers by the inhibition of angiogenesis. Those skilled in the art will be familiar with the various ways of quantifying the responses of the 3DLTC of the present invention.

In another embodiment of the present invention, there is provided a method for the determination of the angiogenesis phenomenon comprising culturing a 3DLTC according to the invention with suitable nutrients for a time sufficient to allow growth of new vascular tissue, and examining the 3DLTC to determine whether new vascular tissue has grown or not.

Several substances, or combination of substances, which are considered as potentially significant molecules for eliciting an angiogenesis modulation activity, may be screened by the present method. This may include, but not limited to, purified preparations of compounds and various extracts from plants, animals or microorganisms. Accordingly, such substances may have to be prepared into a suitable form for administration to the 3DLTC. Those skilled in the art will be familiar with various methods for formulating such substances into suitable form for administration.

One particular embodiment of the present invention is to provide a 3DLTC comprising cancer cells at different levels of malignancy, and a method of assessing the effect of different compounds and agents on a 3DLTC comprising these various types of cells. The 3DLTC of the present invention comprising cancer cells has the advantage to be representative of the cells' communication and interaction with one another and the extracellular matrix similar to those happening in in vivo conditions. In this context, the compounds or agents that are assessed, react more specifically and more representatively on the different type of cells of the 3DLTC.

The present invention will be more readily understood by referring to the following example, which is given to illustrate the invention rather than to limit its scope.

### EXAMPLE I

### In Vitro Production Capillary Network

Material and Methods

Cell Isolation

Keratinocytes and dermal fibroblasts were obtained from a breast reductive surgery. Briefly skin strips (2 by 10 mm) were incubated overnight in a cold thermolysin solution (500 μg/mL in an HEPES solution) (Sigma, Oakville, Canada). The epidermis was peeled from the dermis and incubated 30 minutes in a trypsin-EDTA solution (0.05% trypsin, 0.01% EDTA) (Sigma) to dissociate the epithelial cells. The dermis was incubated three hours in a collagenase H solution (0.125 U/mL in the fibroblast culture medium) to recuperate the fibroblasts.

Endothelial cells were obtained from the digestion of an umbilical cord vein with thermolysin. Briefly, a warm solution of thermolysin (250 μg/mL in an HEPES solution) (Sigma) was introduced in the vein's lumen and kept in place for 60 minutes. Cells were recovered by perfusion.

Premalignant and malignant cell lines were purchased from the American Type Culture Collection (Manassas, USA). The cell lines used were derived from a mammary gland adenocarcinoma (MCF-7), a breast infiltrating ductal carcinoma (BT 549), and a metastatic lymph node of a vulvar carcinoma (SW 962). Cells were cultivated in Dulbecco-Vogt modification of Eagle's medium (DME, Life Technologies, Grand Island, USA) containing 10% foetal calf serum (FCS, Hyclone, Logan, USA), 100 IU/mL penicillin G (Sigma), and 25 μg/mL gentamycin (Sigma). Insulin (5 μg/mL, Sigma) was added to the culture medium of the BT 549 cell line.

Production of the In Vitro Model

Dermal fibroblasts were seeded at 12 000 cells per square centimetre and cultivated in fibroblast medium (Dulbecco-Vogt modification of Eagle's medium containing 10% FCS (Hyclone), 100 IU/mL penicillin G (Sigma), and 25 μg/mL gentamycin (Sigma)) containing 50 μg/mL of ascorbic acid (sodium ascorbate, Sigma) for four weeks to form manipulatable sheets. Monocultures of endothelial cells were cultured in an endothelial cell medium (M199 medium (Sigma) supplemented with 20% FCS (Hyclone), 20 μg/mL Endothelial Cell Growth Supplement (Sigma), 40 U/mL heparin (Leo laboratories, Ajax, Canada), 1.6 mM/mL glutamin (Sigma), 100 IU/mL penicillin G (Sigma), and 25 μg/mL gentamycin (Sigma). Endothelial cells were added (12 000 cells per square centimetre) on the fibroblasts' sheets and cultivated for an additional week in a mixed cell medium (a 1:1 mixture of fibroblast medium and endothelial cell medium) containing 50 μg/mL of ascorbic acid. Two of these sheets were then superimposed to form a reconstructed vascularized stroma. After a week of maturation in the mixed cell medium, the reconstructed stroma was seeded with epithelial cells at a density of 250 000 cells per square centimetre. This reconstructed stroma and epithelium was cultivated for another week under submerged conditions in the epithelial cell medium (DME with Ham's F12 in a 3:1 proportion, supplemented with 5% FCS (Hyclone), 10 ng/mL epidermal growth factor (Austral biologicals, San Ramon USA), 24.3 μg/mL adenin (Sigma), 5 μg/mL insulin (Sigma), 5 μg/mL transferrin (Sigma), 2×10⁻⁹ M 3,3′5′ triiodo-L-thyronin (Sigma), 0.4 μg/mL hydrocortisone (Calbiochem, La Jolla, USA), 0.10⁻¹⁰ M cholera toxin (ICN biomedicals, Costa Mesa, USA), 100 IU/mL penicillin G (Sigma), and 25 μg/mL gentamycin (Sigma). This cell system was then brought to the air-liquid interface and cultivated with the air-liquid medium (epithelial cell medium without the epidermal growth factor) for up to three weeks. Biopsy samples were harvested either at one week under submerged conditions or after one, two or three weeks of culture at the air-liquid interface, then processed for various examinations.

Histological Analysis

Biopsies of the in vitro 3DLTC were fixed at least 24 hours in a Bouin. solution (ACP, Canada) and embedded in paraffin. Cross-sections of 5 μm were stained with Masson's trichrome or with Hematoxylin-Phloxine-Safran.

For the tubes counts, forty consecutives cross-sections were mounted onto slides. The cross-sections numbered 1, 10, 19, 28 and 37 were stained with Masson's trichrome. Three pictures were taken at the 40× objective with a digital camera (CoolSnap RS Photometrics™, Roper Scientific, Munich, Germany) for each biopsy (two biopsies per condition). The number of tubes was counted in each field. The fields with the most vascular structure from each cross-section and each biopsy were compiled and submitted to the Student-Newman-Keuls (S.N.K.) test for statistical analysis of significance.

Immunofluorescence Staining

Samples of the in vitro angiogenesis model were also embedded and frozen in OCT compound (Sakura Finetek, United States). Cross-sections of 4 μm of the reconstructed skin were fixed in acetone and incubated 30 minutes at room temperature with different antibodies (Rabbit anti-factor VIII (Sigma), Mouse anti-PECAM (Chemicon™), Rat anti-laminin (Immunotec™), Sheep anti-type IV collagen (Michigan University), Mouse anti-fibrillin (Neomarker™), Mouse anti-fibronectin (ATCC), Mouse anti-type I collagen (Chemicon™), and 10 minutes with a solution of Hoescht 33258™,

Ultrastructural Studies

Biopsies of the in vitro angiogenesis model were fixed in a 0,1 M cocadylate solution containing 0,1% glutaraldehyde. The tissues were then post-fixed in a 1% osmium tetroxide solution, dehydrated in ethanol and embedded in epoxy. Ultrathin sections (60-80 nm) were stained with uranyl acetate and lead citrate and observed with a JEOL 1200 EX™ electron microscope.

Confocal Microscopy Observations

Tissues were fixed 30 minutes in acetone, and incubated with a primary antibody against PECAM (Mouse monoclonal, Chemicon™, Temecula, USA) and a secondary antibody coupled to an Alexa 488™ fluorochrome (Polyclonal Rabbit anti-mouse, Molecular Probes, Eugene, USA). Images were acquired on a BIO-Rad MRC-1024™ equipped with an Argon-Krypton laser and a Nikon Diaphot-TMD™ using a 60×lens.

Results

Features of the In Vitro Cell System Containing Normal (Non-Malignant) Epithelial Cells

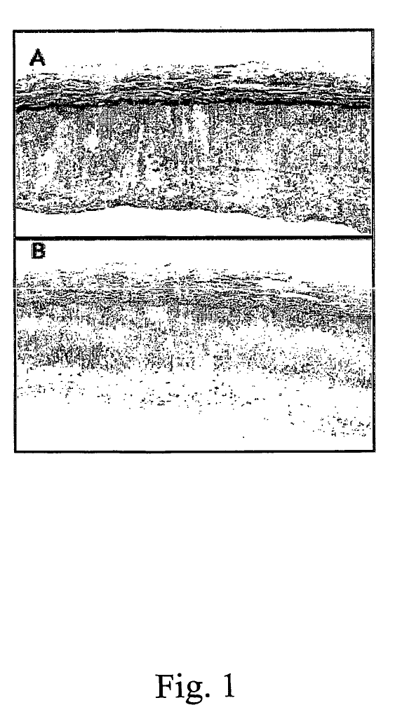

When keratinocytes were seeded onto the reconstructed vascularized stroma and cultivated for three weeks at the air-liquid interface, they fully differentiated into an epidermis (Masson's trichrome (FIG. 1A), and hematoxylin-phloxine-safran staining (FIG. 1B)). Note the presence of tubes in the stromal moiety and the differentiation of the epidermis. The reconstructed epidermis is composed of the four characteristic layers (basal, spinous, granular, and cornified) found in the normal human skin and lays on a basement membrane. The reconstructed stroma presented a dense collagen network (blue colour in the Masson's trichrome stain) and contained hollow circular structures reminiscent of capillaries of the microcirculation.

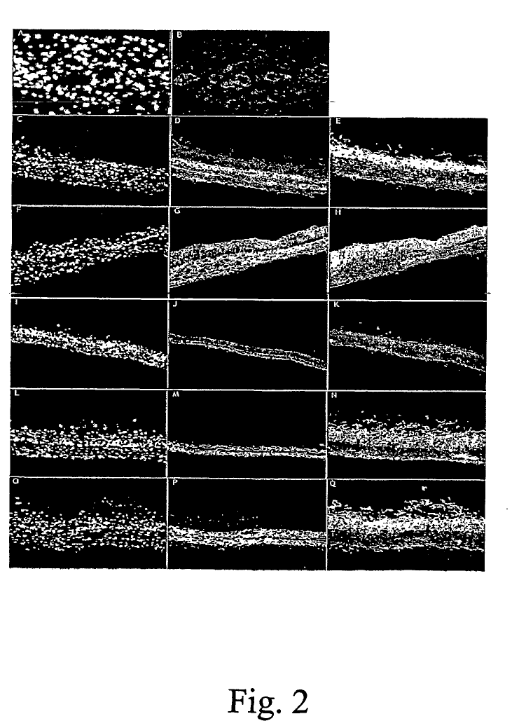

In order to confirm the endothelial nature of the circular structures found in the stromal moiety of the in vitro cell system, immunofluorescence labellings of specific antigens were performed (FIGS. 2A to 2Q). Labelling of endothelial cell markers (Factor VIII) are shown in FIGS. 2E, 2H, 2K, 2N, and 2Q. Platelet-Endothelial-Cell-Adhesion-Molecule (PECAM) is shown in FIG. 2B. Proteins of the basement membrane, laminin are shown in FIG. 2D, and type IV collagen is shown in FIG. 2G. Elastic fibres (fibrillin) are shown in FIG. 2M. Components of the extracellular matrix (fibronectin) can be visualised in FIG. 2J. Type I collagen is shown in FIG. 2P. Nuclei were stained with Hoescht 33258 (FIGS. 2A, 2C, 2F, 2I, 2L, and 20). Cells lining the circular structures stained positively for two specific endothelial cells markers (factor VIII and PECAM) and these cells were associated with one another to form tubes with a lumen. Furthermore, components of the basement membrane (laminin and type IV collagen) were also detected underneath the circular structures suggesting the presence of a basement membrane surrounding the tube. Finally, fibrillin, fibronectin, and type I collagen three components of the dermal extracellular matrix, were also detected in the reconstructed stroma.

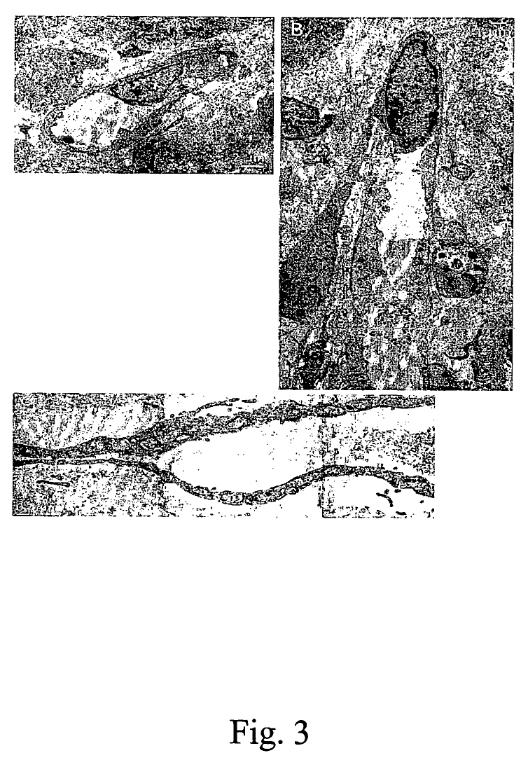

Ultrathin sections of the in vitro angiogenesis 3DLTC of angiogenesis were observed under transmission electron microscopy for the analyses of the structures of the endothelialized tubes (FIGS. 3A to 3C). The endothelialized tubes were observed along their lateral axis with a lumen of 7 μm (FIG. 3A), and also an angiogenic sprout (FIG. 3B), along their longitudinal axis with a lumen of 3 μm (FIG. 3C). Endothelial cells are tightly organized into tubes with a lumen size consistent with those of the capillaries of the human and mammalian microcirculation. An angiogenic sprout, the migration tip of a newly formed blood vessel, has also been observed. This last observation suggests that active angiogenesis can be found and analyzed in the present in vitro 3DLTC.

Tridimensional Analysis of Capillary Network of a In Vitro Tridimensional Living Tissue Construct of Angiogenesis

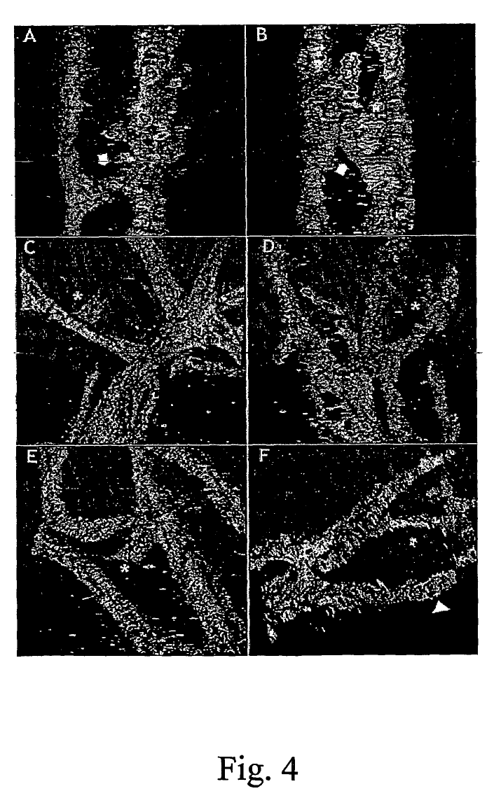

To demonstrate the tridimensional nature of the endothelialized tubes themselves and the presence of a network of tubes, confocal microscopy analysis was performed (FIGS. 4A to 4F) Tridimensional reconstruction of the capillary-like network of the reconstructed skin was labelled with an antibody against PECAM. Note the presence of a junction between two capillary networks (arrows in FIGS. 4A and 4B), an angiogenic sprout (asterisks in FIGS. 4C, 4D, and 4E), and the tube's lumen (triangle in FIG. 4F). Endothelialized structures were labelled with an antibody against PECAM, a surface antigen found on endothelial cells. The tridimensional reconstitution of the PECAM labelled cells showed the presence of hollow tubes (capillaries) that were branched and connected with one another to form a tridimensional network. In the angiogenesis 3DLTC, endothelial cells rearranged into small hollow cylinders. In addition, as previously shown by transmission electron microscopy (FIG. 3B), an angiogenic sprout was also observed, indicating active angiogenesis in the in vitro angiogenesis 3DLTC.

Features of the In Vitro Tridimensional Living Tissue Construct Containing Malignant Epithelial Cells

Since the first part of the present study demonstrated the presence of a tridimensional capillary network (FIGS. 4A to 4F) in the in vitro cell system, premalignant and malignant epithelial cells from various carcinoma were next seeded onto the endothelialized stroma to monitor the effects of these cells on the capillary network. Three different grades of carcinoma (in situ (pre-malignant), infiltrating and a lymph node metastasis) were chosen to study the effect of cancer progression on the capillary network present in the reconstructed stroma.

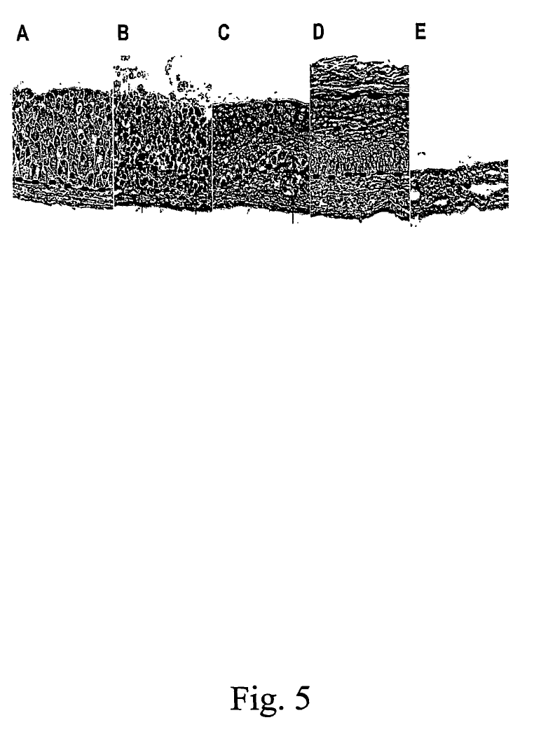

The histological cross-sections demonstrate that each carcinoma cell lines behaved differently on the reconstructed stroma (FIGS. 5A to 5E). Histological properties (Masson's trichrome staining) of the in vitro angiogenesis model containing different types of epithelial cells after 21 days of maturation at the air-liquid interface are shown. Note that the in situ carcinoma (FIG. 5A), infiltrating carcinoma (FIG. 5B), metastatic carcinoma cells (FIG. 5C) behaved quite differently on the reconstructed stroma. As a comparison, a normal epithelium (FIG. 5D) and reconstructed stroma without an epithelium (FIG. 5E) are shown). The in situ carcinoma cells proliferated and piled up to form a multilayered epithelium, but they did not invade the reconstructed stroma or cross the basement membrane. In comparison, infiltrating carcinoma cells were observed to cross the basement membrane and penetrate the reconstructed stroma as clusters of cells migrating into. the extracellular matrix. The metastatic lymph node cells are able to migrate into the collageneous matrix and invade stromal tissues. They can also augment the number of capillaries of the underneath stroma.

In addition, the absence of epithelial cells on the stroma leads to the formation of giant capillaries suggesting an inhibitory role for epithelial cells on the growth of capillaries.

To evaluate the sensitivity of the present 3DLTC for quantitative studies, the number of capillaries on the histological cross-sections of the tissues reconstructed with different normal and malignant epithelial cells were counted and compared (Table 1). The number of capillary tubes present in the tissue reconstructed with metastatic epithelial cells was significantly higher than that of tissues reconstructed with other cell types after seven days of submerged culture or after with a culture at the air-liquid interface. At the two other time points (7 days or 14 days of maturation at the air-liquid interface), there were non-significant differences.

The number of capillaries on the histological cross-sections of the different epithelium was counted and compared. The number of tubes of the metastatic cells system was significantly higher than all other cell types before the maturation at the air-liquid interface (0 days) or after 21 days of maturation.

TABLE 1
Comparison of capillary counts for the different epithelium

| | | | Mean number of |
|---|---|---|---|
| Type of | Days of | | capillaries per field |
| Epithelium | maturation | N | ± S.D. |
| None | 0 | 1 | 14.9 ± 5.8 |
| | | 2 | 19.3 ± 5.9 |
| Normal | 0 | 1 | 19.5 ± 3.7 |
| | | 2 | 15.6 ± 2.9 |
| In situ carcinoma | 0 | 1 | 26.2 ± 7.6 |
| | | 2 | 21.9 ± 3.5 |
| Infiltrating | 0 | 1 | 19.2 ± 4.7 |
| carcinoma | | 2 | 21.4 ± 8.3 |
| Metastasis of | 0 | 1 | 39.3 ± 7.6** |
| a carcinoma | | 2 | 42.4 ± 11.9** |
| None | 21 | 1 | 9.6 ± 3.3 |
| | | 2 | 9.6 ± 2.2 |
| Normal | 21 | 1 | 17.8 ± 3.8 |
| | | 2 | 5,8 ± 1.3 |
| In situ carcinoma | 21 | 1 | 21.3 ± 5.9** |
| | | 2 | 12.6 ± 2.3** |
| Infiltrating | 21 | 1 | 11.3 ± 2.0 |
| carcinoma | | 2 | 8.7 ± 2.6 |
| Metastasis of | 21 | 1 | 34.0 ± 8.2** |
| a carcinoma | | 2 | 16.4 ± 4.1** |

Legend:
N, number of the repetition;
S.D., standard deviation;
**statistically different p < 0.05

Discussion

In the in vitro 3DLTC, the cells secreted and self-assembled the extracellular matrix thus reproducing a tridimensional microenvironment favorable for the development of a capillary vascular network. These observations support that the proper signals (extracellular matrix, diffusible factors from co-cultured cells, etc.) for the maintenance of the integrity of the capillaries were still present after seven (7) weeks after tissue dissection, cell isolation and culture.

The capillary structures present in the. reconstructed skin may be composed of differentiated endothelial cells forming tubes. The ultrastructural observations of the capillaries indicate that they possess a lumen of a physiological size (7 μm), are formed of tightly organized endothelial cells, and that active angiogenesis (sprouting) was present in the reconstructed tissue. Those endothelialized tubes laid on a basement membrane and were embedded in a complex tridimensional extracellular matrix.

Confocal microscopy, as well as electron microscopy, showed the presence of ongoing angiogenesis in the in vitro cell system. In addition, tridimensional reconstruction of the capillary network clearly demonstrated that the capillaries branched into one another forming a complex network distributed in the reconstructed stroma. It was also shown that the tubes presented a clear, and open lumen.

In addition, the epithelium seeded on the endothelialized stroma has an influence in the number of capillaries present. An epithelium formed with metastatic cells of a carcinoma origin elicited the formation of numerous capillaries. Furthermore, the absence of an epithelium leads to the formation of giant capillaries, thereby corroborating the influence of epithelial cells on the normal growth of blood vessels.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

What is claimed is:

1. A tridimensional living tissue construct (3DLTC) comprising fibroblasts and endothelial cells cultured under conditions allowing synthesis and self-assembling of an extracellular matrix, said cultured fibroblasts and endothelial cells being embedded into or associated with said synthetized extracellular matrix leading to the formation of a tridimensional capillary network within said 3DLTC.

2. The tridimensional living tissue construct of claim 1, wherein said extracellular matrix comprising said embedded fibroblasts and endothelial cells is capable of supporting the growth of an epithelial cell type.

3. The tridimensional living tissue construct of claim 1, wherein said fibroblasts and endothelial cells are of human origin.

4. The tridimensional living tissue construct of claim 2, wherein said epithelial cells are derived from a normal, a pre-malignant or a malignant epithelium.

5. The tridimensional living tissue construct of claim 2, wherein said epithelial cell type is of human origin.

6. The tridimensional living tissue construct of claim 1, wherein said fibroblasts and endothelial cells further self-assemble into a stroma.

7. The tridimensional living tissue construct of claim 2, wherein said extracellular matrix comprising said embedded fibroblasts and endothelial cells further comprises an epithelial cell type for forming an epithelium.

8. A method for determining potential angiogenic modulating properties of at least one agent based on measurement of vessels in a capillary network present in a tridimensional living tissue construct (3DLTC) as described in claim 1, comprising the steps of:
a) contacting at least one agent with a 3DLTC in culture, said agent being dissolved in a cell culture medium or applied onto said 3DLTC for a time sufficient to allow said agent to inhibit or stimulate growth of vessels in said 3DLTC; and
b) measuring growth of vessels of said capillary network in contact with said at least one agent,
wherein said measurement of growth comprises determining a growth inhibition or a growth stimulation of said vessels compared to the growth of vessels of a 3DLTC non-treated with said at least one agent.

9. The method of claim 8, wherein said agent is selected from the group consisting of a peptide, a polypeptide, a protein, a fatty acid, a lipid, sugar, a carbohydrate or a combination thereof.

10. The method of claim 8, wherein said tridimensional living tissue construct contains epithelial cells derived from a normal, a pre-malignant or a malignant epithelium.

11. A method for determining potential angiogenic modulating properties of exogenous cells based on growth of vessels in a capillary network present in a 3DLTC as described in claim 1, said method comprising the steps of:
a) contacting said exogenous cell with said 3DLTC, either applied directly onto said 3DLTC or placed in a same culture medium for a time sufficient to allow said exogenous cell to inhibit or stimulate growth of vessels in said 3DLTC; and
b) measuring growth of vessels of the capillary network of said 3DLTC in contact with said cell;
wherein said exogenous cells are exogenous with respect to cells contained in the 3DLTC, and an intrinsic ability of said exogenous cell to affect angiogenesis in vivo is reflected in said 3DLTC by stimulating or inhibiting the angiogenesis.

12. The method of claim 11, wherein said tridimensional living tissue construct contains epithelial cells derived from a normal, a pre-malignant or a malignant epithelium.
